(19) United States
(12) Patent Application Publication
(10) Pub. No.: US 2022/0258142 A1
(43) Pub. Date: Aug. 18, 2022

(54) METALLOSILICATE CATALYST SOLVENT WASH

(71) Applicant: Dow Global Technologies LLC, Midland, MI (US)

(72) Inventors: Wen-Sheng Lee, Midland, MI (US); Le Wang, Pearland, TX (US); Thomas H. Peterson, Midland, MI (US); Sung-Yu Ku, Manvel, TX (US); Wanglin Yu, Pearland, TX (US); Stephen W. King, Braselton, GA (US)

(21) Appl. No.: 17/626,156

(22) PCT Filed: Sep. 29, 2020
(86) PCT No.: PCT/US2020/053201

§ 371 Date: Jan. 11, 2022

Related U.S. Application Data
(60) Provisional application No. 62/908,117, filed on Sep. 30, 2019.

(51) Int. Cl. B01J 29/90; B01J 29/70; B01J 38/56; B01J 38/52; C07C 41/06

(52) U.S. Cl. CPC B01J 29/90; B01J 29/7007; C07C 41/06; B01J 38/52; B01J 38/56

(57) ABSTRACT

A method includes the steps of (a) contacting a solvent having a Water Solubility of 1 g or greater per 100 g of water with a metallosilicate catalyst having an alumina to silica ratio from 5 to 1500; and (b) heating the metallosilicate catalyst to a temperature from 125 C to 300 C fora period of 0.5 hours to 5 hours.

### BACKGROUND

### Field of the invention

The present disclosure generally relates to metallosilicate catalysts and more specifically to the regeneration of metallosilicate catalysts utilizing a solvent wash.

### Introduction

Production of secondary alcohol ethoxylate surfactants can be carried out by the catalyzed ethoxylation of (poly)alkylene glycol monoalkyl ether (“monoalkyl ether”). The monoalkyl ether is formed from an olefin and a (poly)alkylene glycol using metallosilicate catalysts. Metallosilicate catalysts offer a selectivity for monoalkyl ether of greater than 80% which is advantageous as (poly)alkylene glycol dialkyl ether (“dialkyl ether”) are deleterious to properties of the secondary alcohol ethoxylate surfactants.

Although providing greater than 80% selectivity for monoalkyl ether, the metallosilicate catalysts foul quickly resulting in short in-service times, low monoalkyl ether yield and the need for repeated catalyst regeneration steps. Washing catalysts during a catalyst regeneration process has been attempted. For example, regeneration of specific metallosilicate catalysts by washing the catalyst in ethanol followed by drying at 150° C. has been found to be ineffective in restoring catalytic activity.

Accordingly, it would be surprising to discover a solvent wash regeneration process that regenerates metallosilicate catalyst monoalkyl ether production rates and selectivity comparable to a fresh metallosilicate catalyst.

### SUMMARY

The present invention offers a solution to providing a solvent wash regeneration process that regenerates a metallosilicate catalyst monoalkyl ether production rates and selectivity comparable to a fresh metallosilicate catalyst.

The present invention is a result of discovering that washing a metallosilicate catalyst having a silica to alumina ratio of from 5 to 1500 in a solvent having a Water Solubility of 1 gram (g) or greater per 100 g of water during a regeneration process followed by heating the catalyst to a temperature from 125° C. to 300° C. for a time period of 0.5 hours to 5 hours unexpectedly provides a catalyst with a monoalkyl ether production rate and selectivity comparable to a fresh catalyst. Such a result is surprising in that heating the washed catalyst to the boiling point of the wash solvent for an extended period of time has been found to be insufficient to restore catalytic activity, but rather the catalyst must be heated in excess of the boiling point to restore catalytic activity. Further, the restoration of catalytic activity is surprising in view of the failure of the prior art to regenerate catalysts using similar solvent wash techniques.

According to at least one feature of the present disclosure, a method includes the steps of (a) contacting a solvent having a Water Solubility of 1 g or greater per 100 g of water with a metallosilicate catalyst having an alumina to silica ratio from 5 to 1500; and (b) heating the metallosilicate catalyst to a temperature from 125° C. to 300° C. for a period of 0.5 hours to 5 hours.

### DETAILED DESCRIPTION

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

All ranges include endpoints unless otherwise stated.

Test methods refer to the most recent test method as of the priority date of this document unless a date is indicated with the test method number as a hyphenated two-digit number. References to test methods contain both a reference to the testing society and the test method number. Test method organizations are referenced by one of the following abbreviations: ASTM refers to ASTM International (formerly known as American Society for Testing and Materials); EN refers to European Norm; DIN refers to Deutsches Institut fur Normung; and ISO refers to International Organization for Standards.

IUPAC codes describing Crystal structures as delineated by the Structure Commission of the International Zeolite Association refer to the most recent designation as of the priority date of this document unless otherwise indicated.

As used herein, the term weight percent (“wt %”) designates the percentage by weight a component is of a total weight of an indicated composition.

### Method

The method of the present invention is directed to the regeneration of metallosilicate catalysts. The method may comprise steps of catalyzing a reaction of an olefin and an alcohol using the metallosilicate catalyst, generating an alkylene glycol monoalkyl ether, contacting a solvent having a Water Solubility of 1 g or greater per 100 g of water with the metallosilicate catalyst; and heating the metallosilicate catalyst to a temperature from 125° C. to 300° C. for a period of 0.5 hours to 12 hours.

### Olefin

The olefin used in the method may be linear, branched, acyclic, cyclic, or mixtures thereof. The olefin may have from 5 carbons to 30 carbons (i.e., C₅-C₃₀). The olefin may have 5 carbons or greater, or 6 carbons or greater, or 7 carbons or greater, or 8 carbons or greater, or 9 carbons or greater, or 10 carbons or greater, or 11 carbons or greater, or 12 carbons or greater, or 13 carbons or greater, or 14 carbons or greater, or 15 carbons or greater, or 16 carbons or greater, or 17 carbons or greater, or 18 carbons or greater, or 19 carbons or greater, or 20 carbons or greater, or 21 carbons or greater, or 22 carbons or greater, or 23 carbons or greater, or 24 carbons or greater, or 25 carbons or greater, or 26 carbons or greater, or 27 carbons or greater, or 28 carbons or greater, or 29 carbons or greater, while at the same time, 30 carbons or less, or 29 carbons or less, or 28 carbons or less, or 27 carbons or less, or 26 carbons or less, or 25 carbons or less, or 24 carbons or less, or 23 carbons or less, or 22 carbons or less, or 21 carbons or less, or 20 carbons or less, or 19 carbons or less, or 18 carbons or less, or 17 carbons or less, or 16 carbons or less, or 15 carbons or less, or 14 carbons or less, or 13 carbons or less, or 12 carbons or less, or 11 carbons or less, or 10 carbons or less, or 9 carbons or less, or 8 carbons or less, or 7 carbons or less, or 6 carbons or less.

The olefin may include alkenes such as alpha (α) olefins, internal disubstituted olefins, or cyclic structures (e.g., C₃-C₁₂ cycloalkene). α olefins include an unsaturated bond in the a-position of the olefin. Suitable α olefins may be selected from the group consisting of propylene, 1-butene, 1-hexene, 4-methyl-l-pentene, 1-heptene, 1-octene, 1-decene, 1-dodecene, 1-tetradecene, 1-hexadecene, 1-octadecene, 1-icosene, 1-docosene and combinations thereof. Internal disubstituted olefins include an unsaturated bond not in a terminal location on the olefin. Internal olefins may be selected from the group consisting of 2-butene, 2-pentene, 2-hexene, 3-hexene, 2-heptene, 3-heptene, 2-octene, 3-octene, 4-octene, 2-nonene, 3-nonene, 4-nonene, 2-decene, 3-decene, 4-decene, 5-decene and combinations thereof. Other exemplary olefins may include butadiene and styrene.

Examples of suitable commercially available olefins include NEODENE™ 6-XHP, NEODENE™ 8, NEODENE™ 10, NEODENE™ 12, NEODENE™ 14, NEODENE™ 16, NEODENE™ 1214, NEODENE™ 1416, NEODENE™ 16148 from Shell, The Hague, Netherlands.

### Alcohol

The alcohol utilized in the method may comprise a single hydroxyl group, may comprise two hydroxyl groups (i.e., a glycol) or may comprise three hydroxyl groups. The alcohol may include 1 carbon or greater, or 2 carbons or greater, or 3 carbons or greater, or 4 carbons or greater, or 5 carbons or greater, or 6 carbons or greater, or 7 carbons or greater, or 8 carbons or greater, or 9 carbons or greater, while at the same time, 10 carbons or less, or 9 carbons or less, or 8 carbons or less, or 7 carbons or less, or 6 carbons or less, or 5 carbons or less, or 4 carbons or less, or 3 carbons or less, or 2 carbons or less. The alcohol may be selected from the group consisting of methanol, ethanol, monoethylene glycol, diethylene glycol, propylene glycol, triethylene glycol, polyethylene glycol, monopropylene glycol, dipropylene glycol, tripropylene glycol, polypropylene glycol, 1,3-propanediol, 1,2-butanediol, 2,3-butanediol, 1,4-butanediol, 1,6-hexanediol, 1,4-cyclohexanemethanediol, glycerol and/or combinations thereof. According to various examples, the alcohol is a (poly)alkylene glycol such as monoethylene glycol, diethylene glycol, propylene glycol and triethylene glycol.

A molar ratio of alcohol to olefin in the method may be from be 20:1 or less, or 15:1 or less, or 10:1 or less, or 9:1 or less, or 8:1 or less, or 7:1 or less, or 6:1 or less, or 5:1 or less, or 4:1 or less, or 3:1 or less, or 2:1 or less, or 0.2:1 or less, while at the same time, 0.1:1 or greater, or 1:1 or greater, or 1:2 or greater, or 1:3 or greater, or 1:4 or greater, or 1:5 or greater, or 1:6 or greater, or 1:7 or greater, or 1:8 or greater, or 1:9 or greater, or 1:10 or greater, or 1:15 or greater, or 1:20 or greater.

### Metallosilicate Catalyst

As used herein the term “metallosilicate catalyst” is an aluminosilicate (commonly referred to as a zeolite) compound having a crystal lattice that has had one or more metal elements substituted in the crystal lattice for a silicon atom. The crystal lattice of the metallosilicate catalyst form cavities and channels inside where cations, water and/or small molecules may reside. The substitute metal element may include one or more metals selected from the group consisting of B, Al, Ga, In, Ge, Sn, P, As, Sb, Sc, Y, La, Ti, Zr, V, Cr, Mn, Pb, Pd, Pt, Au, Fe, Co, Ni, Cu, Zn. The metallosilicate catalyst may be substantially free of Hf. According to various examples, the metallosilicate may have a silica to alumina ratio of from 5:1 to 1,500:1 as measured using Neutron Activation Analysis. The silica to alumina ratio may be from 5:1 to 1,500:1, or from 10:1 to 500:1, or from 10:1 to 400:1, or from 10:1 to 300:1 or from 10:1 to 200:1. Such a silica to alumina ratio may be advantageous in providing a highly homogenous metallosilicate catalyst with an organophilic-hydrophobic selectivity that adsorb non-polar organic molecules.

The metallosilicate catalyst may have one or more ion-exchangeable cations outside the crystal lattice. The ion-exchangeable cation may include H⁺, Li⁺, Na⁺, Rb⁺, Cs⁺, Mg²⁺, Ca²⁺, Sr²⁺, Ba²⁺, Sc³⁺, Y³⁺, La³⁺, R₄N⁺, R₄P⁺ (where R is H or alkyl).

The metallosilicate catalyst may take a variety of crystal structures. Specific examples of the metallosilicate catalyst structures include MFI (e.g. ZSM-5), MEL (e.g. ZSM-11), BEA (e.g. β-type zeolite), FAU (e.g. Y-type zeolite), MOR (e.g. Mordenite), MTW (e.g. ZSM-12), and LTL (e.g. Linde L), as described using IUPAC codes in accordance with nomenclature by the Structure Commission of the International Zeolite Association.

The crystalline frameworks of metallosilicate catalyst are represented by networks of molecular-sized channels and cages comprised of corner-shared tetrahedral [TO_{4]} (T═Si or Al) primary building blocks. A negative charge can be introduced onto the framework via the isomorphous substitution of a framework tetravalent silicon by a trivalent metal (e.g., aluminum) atom. The overall charge neutrality is then achieved by the introduction of cationic species compensating for the resulting negative lattice charge. When such a charge-compensation is provided by protons, Brønsted acid sites are formed rendering the resulting H-forms of zeolites strong solid Brønsted acids.

The metallosilicate catalysts may be used in the method in a variety of forms. For example, the metallosilicate catalysts may be powdered (e.g., particles having a longest linear dimension of less than 100 micrometers), granular (e.g., particles having a longest linear dimension of 100 micrometers or greater), or molded articles of powdered and/or granular metallosilicate catalysts.

The metallosilicate catalysts may have a surface area of 100 m²/g or greater, or 200 m²/g or greater, or 300 m²/g or greater, or 400 m²/g or greater, or 500 m²/g or greater, or 600 m²/g or greater, or 700 m²/g or greater, or 800 m²/g or greater, or 900 m²/g or greater, while at the same time, 1000 m²/g or less, or 900 m²/g or less, or 800 m²/g or less, or 700 m²/g or less, or 600 m²/g or less, or 500 m²/g or less, or 400 m²/g or less, or 300 m²/g or less, or 200 m²/g or less. Surface area is measured according to ASTM D4365-19.

Metallosilicate catalysts can be synthesized by hydrothermal synthesis methods. For example, the metallosilicate catalysts can be synthesized from heating a composition comprising a silica source (e.g., silica sol, silica gel, and alkoxysilanes), a metal source (e.g., metal sulfates, metal oxides, metal halides, etc.), and a quaternary ammonium salt such as a tetraethylammonium salt or tetrapropylammonium to a temperature of about 100° C. to about 175° C. until a crystal solid forms. The resulting crystal solid is then filtered off, washed with water, and dried, and then calcined at a temperature form 350° C. to 600° C.

Examples of suitable commercially available metallosilicate catalysts include CP814E, CP814C, CP811C-300, CBV 712, CBV 720, CBV 760, CBV 2314, CBV 10A from ZEOLYST INTERNATIONAL™ of Conshohocken, Pa.

### Generating Monoalkyl Ether

Catalyzing the chemical reaction between an olefin and an alcohol using the metallosilicate catalyst results in the generation of an alkylene glycol monoalkyl ether. The alkylene glycol monoalkyl ether may be a (poly)alkylene glycol monoalkyl ether. The chemical reaction between the olefin and the alcohol is catalyzed by the metallosilicate catalyst in a reactor to generate the monoalkyl ether. Various monoalkyl ethers may be produced for different applications by varying which olefin is utilized and/or by varying which alcohol is utilized. Monoalkyl ether are utilized for a number of applications such as solvents, surfactants, and chemical intermediates, for instance.

The reaction of the olefin and the alcohol may take place at from 50° C. to 300° C. or from 100° C. to 200° C. In a specific example the reaction may be carried out at 150° C. Reaction of the olefin and the alcohol may be carried out in a batch reactor, continuous stirred tank reactor, in a continuous fixed-bed reactor or a fluidized bed reactor. In operation of the chemical reaction, the Brønsted acid sites of the metallosilicate catalyst may catalyze the etherification of the olefin to the alcohol through an addition type reaction. The reaction of the olefin and the alcohol produces the monoalkyl ether.

The addition reaction of the olefin to the glycol may form not only monoalkyl ether but also the dialkyl ether. The metallosilicate catalyst may exhibit a selectivity to produce alkylene monoalkyl ether, but not dialkyl ether. The monoalkyl ether selectivity of the metallosilicate catalyst may be 70% or greater, or 75% or greater, or 80% or greater, or 85% or greater, or 90% or greater, or 95% or greater or 99% or greater, while at the same time, 100% or less, or 95% or less, or 90% or less, or 85% or less, or 80% or less, or 75% or less. The dialkyl ether selectivity may be 0% or greater, or 2% or greater, or 4% or greater, or 6% or greater, or 8% or greater, or 10% or greater, or 12% or greater, or 14% or greater, or 16% or greater, or 18% or greater, while at the same time, 20% or less, or 18% or less, or 16% or less, or 14% or less, or 12% or less, or 10% or less, or 8% or less, or 6% or less, or 4% or less, or 2% or less.

A monoalkyl ether yield is calculated by multiplying the amount of olefin conversion by the monoalkyl ether selectivity. The alkylene glycol monoalkyl ether yield may be 10% or greater, or 15% or greater, or 20% or greater, or 25% or greater, or 30% or greater, or 35% or greater, while at the same time, 40% or less, or 35% or less, or 30% or less, or 25% or less, or 20% or less, or 15% or less. Monoalkyl ether yield is a measure of the catalytic activity and selectivity and is a good measure of the production rate of the metallosilicate catalyst.

During the reaction of the olefin and the alcohol, the catalyst becomes fouled. The fouling has the effect of deactivating (i.e., lost etherification activity >50%) the catalyst within hours.

### Contacting the Metallosilicate Catalyst with a Solvent

Regeneration of the metallosilicate catalyst is performed by contacting the metallosilicate catalyst with a solvent followed by heating the metallosilicate catalyst. The contacting of the solvent with the metallosilicate catalyst may be referred to as a “solvent wash.” The solvent has a solubility in water (i.e., a “Water Solubility”) of 1 g or greater per 100 g of water. Water Solubility is measured according to ASTM D1722-09 under 101,325 Pa (1 atmosphere). The solvent may have a solubility in 100 g of water of 1 g or greater, or 2 g or greater, or 5 g or greater, or 10 g or greater, or 15 g or greater, or 20 g or greater, or 25 g or greater, while at the same time, 30 g or less, or 25 g or less, or 20 g or less, or 15 g or less, or 10 g or less, or 5 g or less, or 2 g or less. As defined herein, water is a solvent that has a Water Solubility of 1 g or greater per 100 g of water. Further, it will be understood that solvents miscible in water are encompassed by the definition of having a solubility in 100 g of water of 1 g or greater. The solvent may be selected from the group consisting of water, methanol, ethanol, 1-propanol, 2-propanol, t-butanol, 1,2-dimethoxyethane, acetone, acetonitrile, diethyl ether, ethyl acetate, methyl ethyl ketone, methyl isobutyl ketone, nitromethane, tetrahydrofurane and combinations thereof.

The solvent may be contacted with the metallosilicate catalyst in a variety of manners. For example, the solvent may be sprayed over the metallosilicate catalyst and/or the metallosilicate catalyst may be partially or fully suspended or submerged in the solvent. The solvent may be passed over the metallosilicate catalyst while the metallosilicate catalyst is still within a reactor (e.g., in a continuous fixed-bed reactor or a fluidized bed reactor). In suspended and/or submerged contact between the solvent and the metallosilicate catalyst, agitation (e.g., vortexing and/or shaking) may be applied to the combined catalyst-solvent system.

The solvent may be contacted with the metallosilicate catalyst for 30 seconds or greater, or 1 minute or greater, or 10 minutes or greater, or 20 minutes or greater, or 30 minutes or greater, or 1 hour or greater, or 2 hours or greater, or 3 hours or greater, or 4 hours or greater, or 5 hours or greater, or 6 hours or greater, or 7 hours or greater, or 8 hours or greater, or 9 hours or greater, or 10 hours or greater, or 11 hours or greater, or 12 hours or greater, or 13 hours or greater, or 14 hours or greater, while at the same time, 15 hours or less, or 14 hours or less, or 13 hours or less, or 12 hours or less, or 11 hours or less, or 10 hours or less, or 9 hours or less, or 8 hours or less, or 7 hours or less, or 6 hours or less, or 5 hours or less, or 4 hours or less, or 3 hours or less, or 2 hours or less, or 1 hour or less, or 30 minutes or less, or 20 minutes or less, or 10 minutes or less, or 1 minute or less. During contact of the solvent and the metallosilicate catalyst, one or both of the solvent and metallosilicate catalyst may be at a temperature of 10° C. or greater, or 20° C. or greater, or 30° C. or greater, or 40° C. or greater, or 50° C. or greater, or 60° C. or greater, or 70° C. or greater, or 80° C. or greater, or 90° C. or greater, or 100° C. or greater, or 110° C. or greater, or 120° C. or greater, or 130° C. or greater, or 140° C. or greater, or 150° C. or greater, while at the same time, 160° C. or less, or 150° C. or less, or 140° C. or less, or 130° C. or less, or 120° C. or less, or 110° C. or less, or 100° C. or less, or 90° C. or less, or 80° C. or less, or 70° C. or less, or 60° C. or less, or 50° C. or less, or 40° C. or less, or 30° C. or less, or 20° C. or less.

The solvent and the metallosilicate catalyst may be separated from one another in a variety of manners. For example, the solvent may be evaporated off the metallosilicate catalyst, the metallosilicate catalyst may be separated by centrifugation and/or other separation techniques. Contact and separation of the metallosilicate catalyst and the solvent may be repeated.

### Heating the Metallosilicate Catalyst

After contacting the solvent and the metallosilicate catalyst, a step of heating the metallosilicate catalyst to a temperature of from 125° C. to 300° C. for a period of 0.5 hours to 5 hours is performed. Heating of the metallosilicate catalyst may be carried out in a variety of ovens, furnaces and enclosures. For example, the heating may take place in rotary kilns, box furnaces, fluidized bed furnaces, roller-hearth kilns, enclosures such as tubes comprising a heating element and mesh belt furnaces. The heating of the metallosilicate catalyst may be carried out in a reactor (e.g., in a continuous fixed-bed reactor or a fluidized bed reactor). The heating of the metallosilicate catalyst may be performed in the absence of liquids (i.e., the metallosilicate catalyst may be dried before and/or during the heating). It yet other examples the solvent may be boiled off the metallosilicate catalyst by the step of heating the metallosilicate catalyst.

The heating of the metallosilicate catalyst may be performed in atmospheric oxygen, under an atmosphere which is inert to the catalyst and fouling on the metallosilicate catalyst or under a vacuum. The vacuum may be about 100,000 Pa or less, 50,000 Pa or less, or 10,000 Pa or less, or 5,000 Pa or less. Inert atmospheres may comprise, nitrogen, argon, helium, CO₂, other gases inert to the fouling and/or combinations thereof. Inert atmospheres may comprise the inert component at 60 volume percent (“vol %”) or greater, or 70 vol % or greater, or 80 vol % or greater, or 90 vol % or greater, while at the same time, 100 vol % or less, or 90 vol % or less, or 80 vol % or less, or 70 vol % or less. Volume percent is measured at the regeneration temperature as the percent of volume occupied by inert component divided by the total cavity space that the metallosilicate catalyst is in. Such inert atmospheres may be achieved by passing the inert gas over the metallosilicate catalyst at a constant rate during the heating.

The heating of the metallosilicate catalyst may be carried out at temperature of 125° C. or greater, or 150° C. or greater, or 175° C. or greater, or 200° C. or greater, or 225° C. or greater, or 250° C. or greater, or 275° C. or greater, while at the same time, 300° C. or less, or 275° C. or less, or 250° C. or less, or 225° C. or less, or 200° C. or less, or 175° C. or less, or 150° C. or less.

The heating of the metallosilicate catalyst may be carried out for a time period of 30 seconds or greater, or 1 minute or greater, or 10 minutes or greater, or 20 minutes or greater, or 30 minutes or greater, or 1 hour or greater, or 2 hours or greater, or 3 hours or greater, or 4 hours or greater, or 5 hours or greater, or 6 hours or greater, or 7 hours or greater, or 8 hours or greater, or 9 hours or greater, or 10 hours or greater, or 11 hours or greater, or 12 hours or greater, or 13 hours or greater, or 14 hours or greater, while at the same time, 15 hours or less, or 14 hours or less, or 13 hours or less, or 12 hours or less, or 11 hours or less, or 10 hours or less, or 9 hours or less, or 8 hours or less, or 7 hours or less, or 6 hours or less, or 5 hours or less, or 4 hours or less, or 3 hours or less, or 2 hours or less, or 1 hour or less, or 30 minutes or less, or 20 minutes or less, or 10 minutes or less, or 1 minute or less.

### EXAMPLES

### Materials

Catalyst is a metallosilicate catalysts defined by a BEA structure and having a silica to alumina ratio of 25:1 and a surface area of 680 m²/g, that is commercially available as CP814E from ZEOLYST INTERNATIONAL™ of Conshohocken, Pa.

1-Dodecene is an alpha olefin that is commercially available as NEODENE™ 12 from the SHELL™ group of The Hague, Netherlands.

Monoethylene Glycol is liquid anhydrous ethylene glycol purchased from SIGMA ALDRICH™ having a CAS Number of 107-21-1.

DME is a Dimethoxyethane that is a liquid anhydrous solvent purchased from SIGMA ALDRICH™ having a CAS Number of 110-71-4.

Hexane is a liquid solvent purchased from FISHER CHEMICAL™ having a CAS number of 110-54-3.

Methanol is a liquid anhydrous solvent purchased from SIGMA ALDRICH™ having a CAS Number of 67-56-1.

Diglyme is bis(2-methoxyethyl) ether that is a liquid anhydrous solvent purchased from SIGMA ALDRICH™ having a CAS Number of 111-96-6.

### Test Methods

### Gas Chromatography Samples

Prepare gas chromatography samples by mixing 100 μL of the example with 10 mL of gas chromatography solution that was prepared by addition of 1 mL of hexadecane in 1 L of ethyl acetate. Analyze the sample using an Agilent 7890B gas chromatography instrument. Determine the total amount of 1-dodecene derived species, which includes monoalkyl ether, dialkyl ether and 2-dodecanol, total amount of dodecene, which includes 1-dodecene and all non 1-dodecene other C₁₂ isomers. Table 1 provides the relevant gas chromatography instrument parameters.

Table 1:

| Chromatograph: | Agilent 7890 Series GC | |
|---|---|---|
| Column: | Agilent HP88, 100 m × 0.25 mm × 0.20 um | |
| Detector | FID | |
| Oven: | 50° C.-7 min-6° C./min-260° C.-1 min | |
| Injector: | 250° | C. |
| Detector: | 300° | C. |
| Carrier: | Helium 2.0 mL/min constant flow mode | |
| Split ratio: | 10 | |
| Make-Up: | Nitrogen 25 mL/min | |
| Air: | 400 | mL/min |
| Hydrogen: | 40 | mL/min |
| Inlet Liner: | Restek PN 23305.5 Sky Precision | |
| | Liner with wool | |
| Sample Size: | 1 | μL |

| | |
|---|---|
| GC vial rinsing solvent: | ethyl acetate |

### Time-On-Stream (TOS)

Calculate the TOS of the catalyst by measuring the total the total time the catalyst has been in contact with the monoethylene glycol, 1-dodecene, catalyst and products at temperatures above 60° C.

### Olefin Conversion

Calculate the percent olefin conversion by dividing the total amount of dodecene derived species by the summation of total amount of dodecene derived species and the amount of dodecene. Multiply the quotient by 100.

### Monoalkyl Ether Selectivity

Calculate the percent monoalkyl ether (ME) selectivity by dividing the total amount of monoalkyl ether by the total amount of dodecene derived species. Multiply the quotient by 100.

### Monoalkyl Ether Yield

Calculate the monoalkyl ether yield by multiplying the olefin conversion value by the monoalkyl ether selectivity value.

### Catalyst Activity

Calculate the catalyst activity by dividing the grams of monoalkyl ether produced by the grams of catalyst used and dividing the quotient by the hours of the reaction.

### Sample Preparation

### Fresh Catalyst

Place a portion of the catalyst fresh from the vendor on a ceramic dish and calcine in a box oven with constant air flow at a temperature of 550° C. for 12 hours.

### Spent Catalyst

Load a 300 milliliter (mL) Parr reactor having a heating jacket and controller with 67 g of monoethylene glycol, 62 g of 1-dodecene and 7.5 g of catalyst in powder form. Seal the reactor and heat to 150° C. under 1100 rotations-per-minutes (rpm) agitation from a pitch blade impeller for 3.5 hours. Remove the contents of the reactor and isolate the catalyst via centrifugation using a SORVALL™ legend X1R centrifuge from THERMO SCIENTIFIC™. Repeat four times to generate sufficient spent catalyst. Transfer the spent catalyst to four ceramic dishes and dry the spent catalyst in a box oven with constant air flow at 105° C. for 8 hours. Grind the dried and spent catalyst into powder using a mortar and pestle. Place the powdered catalyst in a bottle to create the single source of dried, spent catalyst.

### Catalyst Solvent Wash

Load 1.5 g of the dried spent catalyst with 40 ml of the solvent in a 50 mL centrifuge tube at 23° C. Suspended the catalyst via a vortex for 1 minute using a K-550-G vortex mixer from VWR™. Shake the catalyst and solvent for 30 minutes using a Junior Orbit shaker from LAB-LINE INSTRUMENTS™ Inc. Isolate the catalyst via centrifugation with the supernatant solvent decanted. Repeat two more times. Split the washed catalyst into two portions for testing. Heat one portion of the sample to the indicated temperature for the indicated number of hours (H).

### Sample Testing

Test etherification activity using 40 mL vial reactors and rare earth magnetic stir bars set to tumbling stirring. Load the reactors with 0.2 g of catalysts, 6.2 g of 1-dodecene, and 6.7 g of monoethylene glycol. Heat the reactor to 150° C. for 1 hour.

### Results

Table 2 provides the sample testing results for Comparative Examples 1-8 (“CE1-CE8”) and Inventive Examples 1-3 (“IE1-IE3”). Table 2 provides data on olefin conversion, monoalkyl ether selectivity (“ME selectivity”) and monoalkyl ether yield (“ME yield”). CE1 is a fresh sample while CE2 is a sample of the spent catalyst. CE3-CE6 represent samples that have undergone solvent wash, but only heated to 105° C. while IE1-IE3 utilize a solvent wash and are heated to 165° C. CE8 represents a sample that utilizes a solvent wash with a solvent having a Water Solubility of less than 1 g per 100 g of water, but is heated to 165° C. Water as a solvent has a Water Solubility of greater than 1 g per 100 g of water. Methanol is miscible with water and as such has a Water Solubility of greater than 1 g per 100 g of water. Dimethoxyethane is miscible with water and as such has a Water Solubility of greater than 1 g per 100 g of water. Hexane has a Water Solubility of 0.0014 g per 100 g of water.

TABLE 2

| | | Olefin | ME | ME |
|---|---|---|---|---|
| | | Conversion | Selectivity | Yield |
| Sample | Regeneration Conditions | (%) | (%) | (%) |
| CE1 | Fresh | 12.1 | 94 | 11.4 |
| CE2 | Spent | 4.2 | 97 | 4.1 |
| CE3 | Water, 105° C., 3 H | 6.0 | 97 | 5.8 |
| CE4 | Methanol, 105° C., 3 H | 6.4 | 97 | 6.2 |
| CE5 | DME, 105° C., 3 H | 5.7 | 97 | 5.5 |
| CE6 | Hexane, 105° C., 3 H | 5.3 | 98 | 5.2 |
| CE7 | Unwashed, 165° C., 3 H | 3.7 | 98 | 3.6 |
| IE1 | Water, 165° C., 3 H | 11.0 | 91 | 10.0 |
| IE2 | Methanol, 165° C., 3 H | 10.8 | 91 | 9.8 |
| IE3 | DME, 165° C. 3 H | 9.2 | 92 | 8.5 |
| CE8 | Hexane, I65° C. 3 H | 5.6 | 97 | 5.4 |

As evident from CE3-CE6 of Table 2, solvent washing of the catalyst by solvents having a Water Solubility less than or greater than 1 g per 100 g of water and heating to 105° C. only marginally increases the production rate of monoalkyl ether as compared to the unwashed catalyst of CE2. IE1-IE3 demonstrate that washing the catalyst in solvent having a Water Solubility of 1 g or greater per 100 g of water followed by heating to within the range of from 125° C. to 300° C. dramatically increases the olefin conversion (i.e., monoalkyl ether production rate) and monoalkyl ether yield to levels comparable to a fresh catalyst. CE8 demonstrates that hexane, having a Water Solubility of less than 1 g per 100 g of water, does not provide the same increased production rate of monoalkyl ether even when heated to the same temperature. In view of the results, it is clear that only metallosilicate catalysts that were both washed in a solvent having a Water Solubility of 1 g or greater per 100 g of water and heated to within the range of from 125° C. to 300° C. exhibit monoalkyl ether production rate and yield comparable to fresh catalysts.

### Fixed-Bed Reaction Testing

Create a fixed bed reactor for testing by loading 1.5 g of catalyst into a 40.64 cm length and 0.64 cm diameter 316 stainless steel tube reactor. Fill remaining space of the reactor with 1 mm quartz chips. Place a piece of quartz wool at each of the combined catalyst and quartz chips. Connect a reactant feed line to the reactor and provide pumping force using a model 307 Gilson™ single-piston pump at a flow rate of 0.1 ml/min to 0.2 mL/min. Heat the reactor to a temperature of 135 C in its reaction zone and keep pressure in the reactor at 101,325 Pa (1 atmosphere). Mix a reactant feed consisting of 60 g of 1-Dodecene, 60 g of monoethylene Glycol and 300 g of diglyme solvent into a single-phase mixture. Orient the direction of the reactor such that the reactant feed flows down through the reactor. Start the reactant feed flow and run the reactor for the designated time on stream.

Regenerate the catalyst within the reactor by first stopping the reactant feed and then lowering the reactor temperature to 80° C. Purge the reactor with 50 mL/min of N₂ for 0.5 hours. Wash the catalyst with water at a feeding rate of 1 mL/min for 150 minutes. Stop the water feed. Increase the reactor temperature to 180° C. while purging the reactor with N₂ at 50 standard cubic centimeters per minute to dry the catalyst for 2 hours. Lower the reactor temperature to 135° C. and resume reactant feed to resume the reaction.

### Fixed-Bed Reaction Results

Table 3 provides the results of the fixed-bed reaction testing. The catalyst regeneration step was performed at a time on stream of 630 hours.

TABLE 3

| Time on | Olefin | | Catalyst | relative |
|---|---|---|---|---|
| stream | conversion | ME | Activity | rate |
| (h) | (%) | Selectivity | (1/h) | (%) |
| 1 | 30 | 84 | 0.37 | 100 |
| 5 | 30 | 85 | 0.37 | 101 |
| 7 | 38 | 86 | 0.47 | 128 |
| 8 | 29 | 86 | 0.36 | 99 |
| 26 | 24 | 87 | 0.31 | 83 |
| 49 | 22 | 88 | 0.28 | 75 |
| 81 | 19 | 89 | 0.25 | 68 |
| 101 | 18 | 89 | 0.23 | 62 |
| 128 | 16 | 89 | 0.21 | 56 |
| 146 | 15 | 89 | 0.19 | 53 |
| 169 | 14 | 89 | 0.18 | 49 |
| 176 | 13 | 89 | 0.17 | 46 |
| 203 | 12 | 90 | 0.16 | 44 |
| 249 | 11 | 89 | 0.14 | 38 |
| 293 | 9 | 88 | 0.12 | 32 |
| 336 | 8 | 86 | 0.11 | 29 |
| 367 | 8 | 86 | 0.10 | 26 |
| 480 | 8 | 86 | 0.09 | 26 |
| 535 | 7 | 85 | 0.08 | 23 |
| 557 | 6 | 85 | 0.08 | 22 |
| 629 | 5 | 83 | 0.06 | 17 |
| 630 | 26 | 87 | 0.33 | 91 |
| 631 | 25 | 88 | 0.32 | 86 |
| 632 | 24 | 88 | 0.30 | 83 |
| 633 | 23 | 88 | 0.29 | 80 |

As evident from Table 3, regeneration of the catalyst within the fixed-bed reactor by first contacting the catalyst with water followed by heating the catalyst to a temperature of from 125° C. to 300° C. for a period of 0.5 hours to 5 hours greatly increased the catalyst activity (i.e., the monoalkyl ether production rate) from 0.06 1/h to 0.331/h. Regeneration of the catalyst without removal from the fixed bed reactor is particularly advantageous in that the reactor does not need to be disassembled, the reactant feed can be replaced with the solvent used for the solvent wash and the reactor can be used to dry the catalyst.

1. A method, comprising the steps:
(a) contacting a solvent having a Water Solubility of 1 g or greater per 100 g of water with a metallosilicate catalyst having an alumina to silica ratio from 5 to 1500; and
(b) heating the metallosilicate catalyst to a temperature from 125° C. to 300° C. for a period of 0.5 hours to 12 hours.

2. The method of claim 1, further comprising the step:
catalyzing a reaction of an olefin and an alcohol using the metallosilicate catalyst.

3. The method of claim 2, wherein the olefin comprises a C₁₂-C₁₄ olefin.

4. The method of claim 2, wherein the alcohol is selected from the group consisting of monoethylene glycol, diethylene glycol, glycerol and combinations thereof.

5. The method of claim 2, further comprising the step of:
generating an alkylene glycol monoalkyl ether.

6. The method of claim 1, wherein the step of heating the metallosilicate catalyst further comprises heating the metallosilicate catalyst to a temperature from 150° C. to 200° C. for a period of 0.5 hours to 5 hours.

7. The method of claim 6, wherein the step of heating the metallosilicate catalyst further comprises heating the metallosilicate catalyst to a temperature from 125° C. to 300° C. for a period of 2 hours to 4 hours.

8. The method of claim 1, wherein the solvent is selected from the group consisting of water, methanol, ethanol, 1-propanol, 2-propanol, t-butanol, 1,2-dimethoxyethane, acetone, acetonitrile, diethyl ether, ethyl acetate, methyl ethyl ketone, methyl isobutyl ketone, nitromethane, tetrahydrofurane and combinations thereof.
